(19) United States
(12) Patent Application Publication
(10) Pub. No.: US 2018/0000394 A1
(43) Pub. Date: Jan. 4, 2018

(54) METHOD AND SYSTEM FOR CREATING A DIAGNOSTIC VASCULAR WINDOW

(71) Applicant: Fresenius Medical Care Holdings, Inc., Waltham, MA (US)

(72) Inventors: Michael Black, Layton, UT (US); Louis L. Barrett, West Point, UT (US); Peter Kotanko, New York, NY (US); Robert Kossmann, Sante Fe, NM (US)

(21) Appl. No.: 15/639,569

(22) Filed: Jun. 30, 2017

Related U.S. Application Data
(60) Provisional application No. 62/357,184, filed on Jun. 30, 2016.

(51) Int. Cl. A61B 5/1455; A61B 5/145; A61M 1/10; A61M 39/02

(52) U.S. Cl. CPC A61B 5/14557; A61M 39/0247; A61B 5/1455; A61B 5/14535; A61M 1/1086; A61M 2039/0258; A61M 2039/0273

(57) ABSTRACT

Embodiments of the disclosure provide a method and system for providing a diagnostic vascular window that may be used in real time to monitor a patient's fluid conditions in a variety of settings. The diagnostic vascular window may, for example, be used pre-surgery, during surgery, and post-surgery to determine whether a correct type and dose of diuretic drugs are being used for the patient. The diagnostic vascular window utilizes low flow accesses to view blood/fluids leaving and entering the patient's body. In addition, the same amount of fluids leaving the body enters the body, so there are no fluid losses or gains within the diagnostic vascular window. The low flow accesses in conjunction with a monitoring system allows for real-time measurements of blood parameters without fluid loss.

### CROSS-REFERENCE TO RELATED APPLICATIONS

This application claims the benefit of U.S. Provisional Application No. 62/357,184, filed on Jun. 30, 2016, which is hereby incorporated by reference in its entirety.

### BACKGROUND

Blood parameters in post-surgical and in other patients requiring critical care can be used by health professionals to assess a patient's immediate condition. Current practice is to order blood sample draws from the patient and send them to the laboratory for analysis. Limitations exist in how much blood can be removed from critically ill and, often, anemic patients. Further, such blood draws only produce “snapshots” in time when the blood is drawn as to the condition of the patient at that point in time.

Patient conditions in critical care are often fluid and dynamic. Deducing patient condition from periodic blood sampling could miss one or more critical variation if occurring between blood samples. And, samples can be at extended time intervals because patients in critical care environments are there due to serious conditions and likely cannot afford to lose the blood volume associated with frequent blood sample draws.

### SUMMARY

An embodiment of the disclosure provides a method for creating a diagnostic vascular window to monitor a patient's blood in real time. The method includes: (a) installing low flow accesses to the patient's blood vessels, the low flow accesses comprising an arterial side access and a venous side access; (b) attaching a monitoring system to the low flow accesses; (c) starting blood flow to the monitoring system, the blood flowing from the arterial side to the venous side; and (d) measuring blood constituents from blood flowing through the monitoring system, wherein, during a monitoring period, a volume of fluid that flowed out of the arterial side access is equal to a volume of fluid that flowed into the venous side access.

Another embodiment of the disclosure provides a system for monitoring a patient's blood in real time. The system comprises: (a) a blood pump configured to pump blood from an arterial side access to a venous side access, the arterial side access and the venous side access being low flow accesses; (b) tubing coupled to the blood pump, the tubing configured to carry extracorporeal blood from the arterial side access to the venous side access at a flowrate determined by the blood pump; and (c) a blood sensor system coupled to the tubing, the blood sensor system configured to measure blood constituents of the extracorporeal blood flowing through the tubing; wherein the system is configured such that, during a monitoring period, a fluid volume that flowed from the arterial side access is equal to a fluid volume that flowed into the venous side access.

### BRIEF DESCRIPTION OF THE DRAWINGS

The present invention will be described in even greater detail below based on the exemplary figures and embodiments. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 illustrates a high level system diagram of a monitoring system in an exemplary environment;

FIG. 2 illustrates an example embodiment for a surgical and/or Intensive Care Unit (ICU) application of the high level system diagram shown in FIG. 1;

FIG. 3 is a perspective illustration of one of the viewing sides of one embodiment of a low flow optical blood chamber;

FIG. 4 illustrates an example embodiment of an optical sensor clip assembly installed on an example embodiment of a low flow optical blood chamber;

FIG. 5 illustrates an exemplary diagram of a monitor and its interfaces with function of the interfaces under software control; and

FIG. 6 illustrates an example of a process performed with the monitoring system for monitoring blood volume during a surgery procedure with follow-up in a post-surgical environment such as in the ICU.

### DETAILED DESCRIPTION

In some medical situations, real-time access to a patient's blood stream is desirable in order to monitor specific blood parameters. Embodiments of the disclosure provide for real-time access to a patient's blood stream.

Conventional methods and devices have not provided a way to pull extracorporeal blood into a blood lab for continuous measurement of key blood constituents. Conventional blood draws and monitoring are invasive and are not conducted in real time. Due to its limitations, conventional blood draws are sometimes not conducted at all. For example, for patients in an intensive care unit (ICU), to obtain information about hematocrit and/or hemoglobin, a patient's blood is typically drawn into a test tube and laboratory analysis is performed thereon. If the patient cannot tolerate a pulse-oximeter on their finger or toe, a similar blood draw may be performed and measured on a CO-oximeter located in the ICU. This process may require that the CO-oximeter be in the ICU, and it may also dictate very careful handling of the blood sample because movement and agitation of blood samples causes immediate oxygen changes. Therefore, any oxygen measurement must be in close proximity to the patient.

There are also situations where medical practitioners are relying solely on guesswork/estimation with respect to blood characteristics. For example, in cases when an organ replacement is performed, an anesthesiologist may be required to inject the patient with a number of pharmaceuticals and/or solutions to keep the patient stable. The injections add blood volume, and this blood volume must be later removed by the medical practitioner via the administration of diuretics in order to place the patient's blood volume after transplant within a given tolerance of the patient's original blood volume. Some medical opinions indicate that if the original blood volume is not substantially achieved that the success of an organ transplant can be in jeopardy.

Embodiments of the disclosure provide an advanced hemodynamic monitoring method and system that clinical staff and physicians can use to monitor blood constituents and parameters in a continuous manner. The system may be attached to a patient through use of a commonly used venous Peripherally Inserted Central Catheter (“PICC” or “PIC”) line. A small amount of blood is pumped out of the patient through the “arterial” side of the PIC line, routed through a single use, sterile blood chamber and back into the patient through the “Venous” side of the PIC line. By using this technique, a sample of the patient's circulatory system is extended outside the body where observations can be taken without incurring any blood loss. Though a number of sensor systems (acoustical, ultrasonic, optical, etc.) can be interfaced to this extracorporeal blood loop, in a preferred embodiment, an optical sensor which views the blood through a uniform, single use blood chamber for continuous constituent measurements using different wavelengths of light is used. Since blood is circulated from and back into the patient's body, there is no blood loss as in conventional methods where blood samples are extracted from the patient and taken to a lab. Additionally, selected blood parameters are monitored continuously, allowing for observation of dynamic changes in the patient's condition. A monitoring system according to some embodiments of the disclosure can facilitate guided hemodynamic interventions required to stabilize patients and optimize outcomes. In some embodiments, the system can measure at least real-time hematocrit (HCT) and oxygen saturation (SAT). Based on the HCT measurements, change in blood volume (BV) and hemoglobin (Hb) can be calculated and displayed. Other blood parameters (e.g. platelets, carboxyhemoglobin, etc.) can be measured by introduction of additional wavelengths with the attendant calibrations.

A system according to some embodiments of the disclosure may be used in the detection of loss of fluid from a patient's intravascular compartment into the patient's interstitial compartment and third spaces (e.g., the peritoneal cavity and gut lumen). The loss of fluid occurs in many medical situations (e.g., postoperative period of abdominal surgery, liver cirrhosis, congestive heart failure, intestinal ischemia). Loss of fluid from the intravascular compartment into the interstitial compartment and third spaces is also a major component of sepsis. As a result, septic patients require large volumes of replacement fluid in order to maintain their intravascular blood volume. The system may monitor blood volume changes in real time, allowing for correct diagnosis of the fluid changes and facilitating the clinical decisions on how to treat the patient.

A system according to some embodiments of the disclosure may also be used where the opposite situation can be problematic. For example, infusion of drugs and other fluids based on anesthesia practices during surgery can add an unquantified blood volume to the patient. Some studies have shown that in the case of transplant surgery that patient blood volume which departs significantly from the blood volume at the commencement of surgery may place the transplanted organ in jeopardy. Determining the correct type and dose of diuretic drugs is a challenge, and there is currently no simple way to evaluate the overall effect on the patient's blood volume other than estimating the fluid amount added during the procedure and then monitoring urine output afterward.

Embodiments of the disclosure may also be used for hemodynamic monitoring during treatment and care in other situations where hemodynamic compromise is present. Examples of these situations include shock due to hypovolemia, trauma, heart failure, neurogenic shock and acute myocardial infarction (MI) with cardiogenic shock. Hemodynamic monitoring may also benefit situations of increased metabolic demands, requiring increased blood-flow and perfusion, for example, sepsis, burns, major surgery, including pre, intra, and post-operative. These example situations call for efficient clinical decision making taking into account rapid hemodynamic fluctuations which is lacking in conventional approaches, for example, blood sample draw, blood gas meters, and so on.

Current hemodynamic monitoring places an emphasis on a patient's pulse and blood pressure. Blood pressure can relate to perfusion to the brain and the heart. However, it does not help define perfusion to renal and mesenteric beds. Additionally, coronary and cerebral ischemia blood pressure thresholds are variable. The patient's pulse and blood pressure does not capture enough information in dealing with the above identified situations where clinicians need to make, revisit and modify decisions on such things as fluids resuscitation, dosages of cardiac agonists, peripheral vascular acting agents, for example, pressors, and diuretics. These decisions can influence the incidence of complications, duration of ventilation, a requirement for interventions (for example, hemodialysis and chemotherapy), the use of continuous renal replacement therapy (CRRT), the length of hospital stay and even mortality rates.

The following example embodiment of a blood monitoring system in this present disclosure provides for non-invasive (other than a standard PIC line use) and real-time monitoring of blood characteristics thereby avoiding a need for successive, invasive blood draws (particularly for ICU blood monitoring) and eliminating guesswork from blood volume adjustment procedures.

The ICU environment is used as an example since in many cases an ICU patient is already anemic, therefore, lacking in red blood cell volume which is the primary carrier of oxygen to the body and vital organs. Conventional blood draws result in removal of red blood cell volume as one of the constituents in the blood sample. Therefore, the number of blood draws are limited in such a patient because he or she may not be able to tolerate any red blood cell volume loss. The normal regeneration of red cell volume in a healthy patient usually spans several weeks. This regeneration rate limits the number of blood samples that may be obtained from an ICU patient, and therefore, limits the resolution of the patient's blood profile. In conventional blood draw monitoring, any dynamic occurrence in the blood (from an occurrence of spontaneous internal bleeding to an expected reduction in blood volume due to prescribed diuretic drugs) can only be approximated with limited samples or such dynamics can be missed entirely.

Embodiments of the disclosure increase resolution of a patient's blood profile by recirculating the patient's blood, thus requiring no blood draw or loss. Further, the circulating blood can be observed continuously in real time to monitor various blood parameters of interest. As such, a diagnostic vascular window is created for measuring constituents and parameters in a patient's blood.

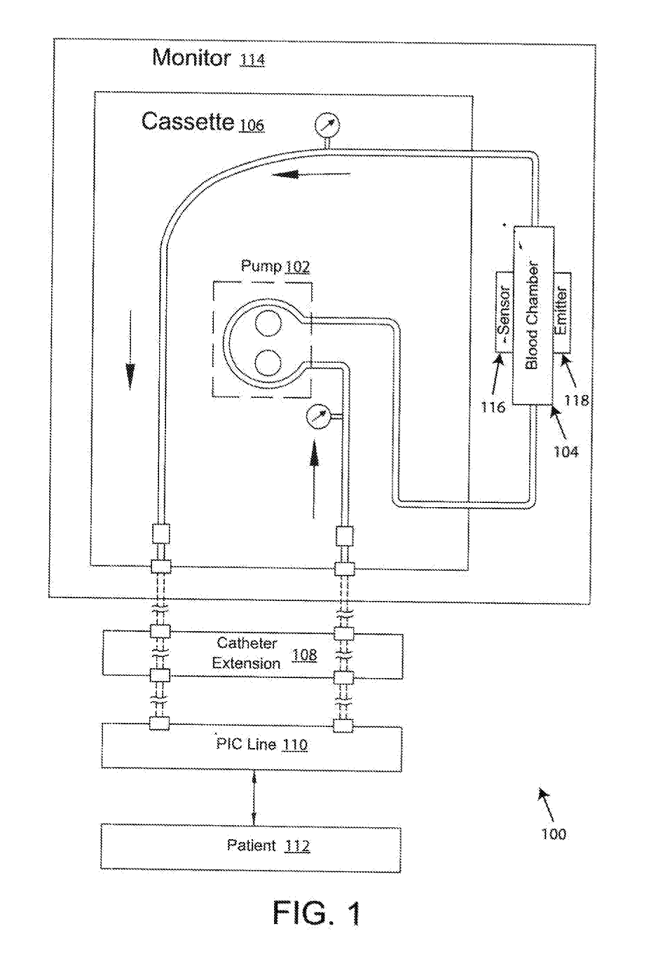

FIG. 1 illustrates a blood composition monitor 114 or monitoring system in an exemplary environment 100 usable with exemplary embodiments of the disclosure. The illustrated environment 100 may be in the ICU, surgery suite, recovery room, or any place examination of a patient's real-time blood condition is deemed valuable for clinical diagnostics. A pump 102 creates the extracorporeal blood flow through the blood chamber 104. In this illustrated embodiment, the pump 102 engages a cassette 106 that includes inlet and outlet blood flow lines for coupling to the blood chamber 104 on one side of the cassette 106 and to a catheter extension line set 108 leading to a PIC line 110 inserted into the patient 112. The monitor 114 receives the cassette 106 such that the inlet line from the arterial side of the catheter extension lines 108 connects with the pump 102 to draw blood from the patient's PIC line 110 to the input of the blood chamber 104 (bottom in FIG. 1). The output of the blood chamber 104 (top in FIG. 1) connects to a return line back through the cassette 106 and through the venous side of the extension lines 108 for returning the blood to the patient 112 through the venous side of the PIC line 110. The catheter extension lines allow the remote blood connection of the monitor system 114 to the PIC line 110 in the patient 112.

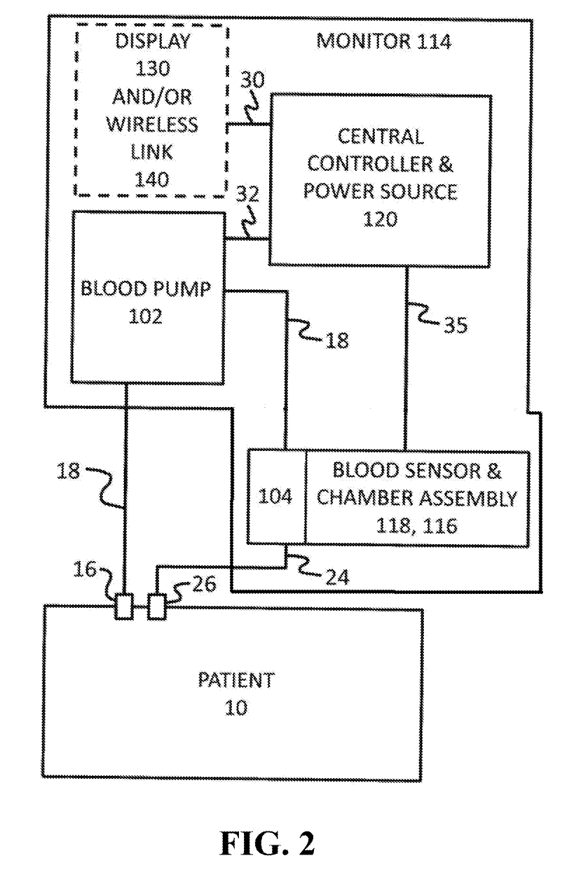

FIG. 2 illustrates additional details of an exemplary embodiment of the overall system shown in FIG. 1. Beginning at the patient 10, an arterial (input) blood connection to the monitor system 114 is provided. In this example, the arterial line 18 is connected to the arterial side of a PIC line inserted into the patient 10. Connections 16 and 26 are the arterial and venous sides of the PIC line, respectively. Blood is pulled from the patient 10 via arterial line 18 by the pump 102. Arterial line 18 continues after the pump to the input of an optical, single use blood chamber 104 and then the blood returns to the patient 10 through venous line 24 to the connection 26 on the venous side of the PIC line.

Unlike dialysis accesses where the patient requires a surgical procedure to implant a shunt (often made of Gore-Tex®) or to grow a thickened vein structured termed a fistula where needle access is frequently (typically three times per week) inserted into the patient, the PIC line is inserted for short term treatment associated with a single surgery or procedure. In dialysis use, the extracorporeal blood circuit is primarily used for dispensing treatment through filtering the blood of impurities. And in dialysis use, blood flowrates found in shunts are upward of 1 liter per minute and high pressures associated with such flowrates are common and must be dealt with. Dialysis uses high flowrates since all blood circulating through a patient needs to be filtered, as such, all of the patient's blood is pumped out while recirculating it for filtering. In contrast to a dialysis access, in the short term treatment, a simple sample loop of the patient's blood coming from a lower flow vein without significant pressure provides an observation window to the core body functions as indicated by changes in blood constituents observed in real time. Accesses for creating a diagnostic vascular window according to embodiments of the disclosure are different from those used during dialysis. Dialysis accesses are punctured with significantly sized needles to support the high blood flow (upwards of 500 milliliters per minute in the United States). Veins or PIC lines are not used as accesses in dialysis since repeated puncturing may damage the access. The diagnostic window does not need to have all the patient's blood pumped out (only a sample), therefore, a low flow rate is capable of being used with the monitoring system 114.

Other examples of low flow accesses exist, such as, that used for assisting temporary or partial kidney failure with CRRT. CRRT is a slow dialysis treatment often given in the ICU. Another example of a low flow access is that used for treating congestive heart failure, such as, accesses used with the Aquadex FlowFlex® system. The CRRT and Aquadex FlexFlow® examples dispense one or more treatments rather than act as a window into a patient's blood system. In these examples where low flow accesses are used, treatments are administered once blood is pulled from the body, thereby providing one or more ways where a patient may gain or lose fluid. In contrast to embodiments of the disclosure, treatment is not administered, thus the amount of fluid exiting the arterial side of an access is the same amount of fluid entering the venous side of an access.

In one example, a low flow venous access supports blood flowrates between 5 milliliters per minute and 50 milliliters per minute. A lower limit is placed on the blood flow rate based on concerns of blood coagulation if blood flow is too low. In another example, a low flow access supports blood flowrates between 10 milliliters per minute and 20 milliliters per minute. These low flow rate examples when contrasted with high flow rates upward of 500 milliliters per minute of arterial blood during dialysis do not have similar risks associated. As already described, the high flow rates of dialysis introduce high pressures that require special accesses that support large needles to support such blood flow. In addition, a human body has about 5 L to 6 L of blood, so when complications arise and a dialysis access needle is pushed out, the patient is at risk to bleed out quickly. In contrast, the low flow access does not deal with such high pressures due to the venous access approach and high flow rates are not used so a patient is not at risk to bleed out if the venous needle becomes dislodged and not observed.

In some embodiments, the PIC line connections 16 and 26 in FIG. 2 providing accesses for blood to be pulled from the patient 10 and returned to the patient 10 may be replaced with two intravenous (IV) needles, strategically placed to feed blood to the measurement blood chamber 104. The blood in the blood chamber 104 can be viewed in real time as part of the patient's circulatory system, and the minimum volume of blood viewed fills the blood chamber 104.

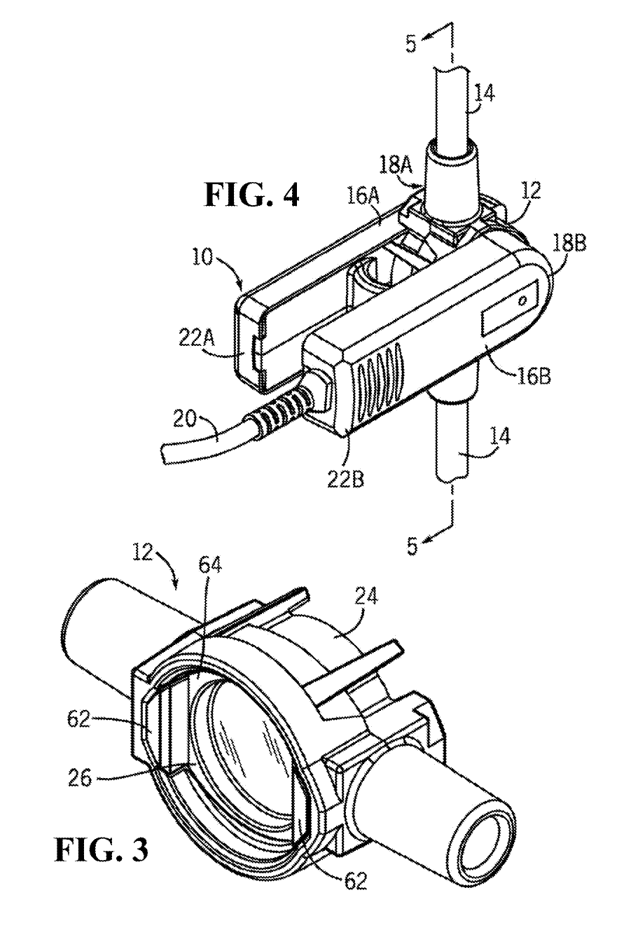

An example of a blood chamber that may be used as the blood chamber 104 is the blood chamber 12 shown in FIG. 3 and disclosed in U.S. Pat. No. 8,333,724 entitled “Low Flow Optical Blood Chamber” which is incorporated by reference in its entirety. The blood chamber 12 may include two molded parts, namely a chamber body 24 and a lens body 26. In one embodiment, the lens body 26 may be sonically welded to the chamber body 24. In another embodiment, the lens body 26 may be secured to the chamber body 24 with medical grade adhesive. Other methods of securing the lens body 26 to the chamber body 24 may be employed provided that the lens body 26 be attached to the chamber body 24 to provide a leak-free blood flow chamber 12. For this reason, there should be sufficient dimensional interference between the lens body 26 and the chamber body 24.

The sensor unit 116 and the emitter unit 118 may be, for example, provided as a single sensor/emitter assembly. In some embodiments, the sensor unit 116 is a photosensor 116 and the emitter unit 118 is a light emitter 118. The physical mounting and mating of the blood chamber 104 and the photosensor 116 and the light emitter 118 can be, for example, associated with a mounting fixture that is part of a cassette 106. However, the photosensor 116 and the light emitter 118 are usually not disposable or manufactured to be disposable, and therefore, are intelligent enough to hold calibration information of parts of a disposable cassette 106.

In one embodiment, the blood chamber 104 and the photosensor 116 and the light emitter 118 interface is as provided by the CRIT-LINE® monitoring system as shown in FIG. 4. The CRIT-LINE® monitoring system approach is disclosed in U.S. Pat. No. 9,173,988 entitled “Sensor Clip Assembly for an Optical Monitoring System” which is incorporated by reference in its entirety. Tubing 14 is attached to the blood chamber 12. The optical sensor clip assembly 10 is an embodiment of the sensor 116/the emitter 118 unit of FIG. 1. In an exemplary embodiment, the tubing 14 is ⅛″ clear, medical grade polypropylene tubing appropriate for use in the peristaltic pump. In an embodiment, the sensor clip assembly 10 includes two arms 16A, 16B forming a spring-biased, jaw-like structure. The handles 22A, 22B on the sensor assembly arms 16A, 16B can be squeezed together against the spring bias to spread the heads 18A, 18B of the sensor assembly to install or remove the sensor assembly 10 on the blood chamber 12.

It will be appreciated that if the monitoring system 114 is optical technology based, the type of photosensor and light emitter can be varied based the blood parameters of interest. For example, the photosensor can be a silicon photodiode with sensitivity in the wavelengths from 500 nm to 900 nm. The light emitter could contain two light emitting diodes (LEDs) of 660 nm and 800 nm which can be measured by the photosensor. If the two LEDs are alternately measured at a fast rate (e.g. 300 times per second per wavelength) then Beer's Law can be used to extract the molar concentration of both oxygenated hemoglobin (660 nm) and isobestic hemoglobin (800 nm). The ratio of these two concentrations allow the hemoglobin term to divide out and leave only the oxygen content of the blood. A calibration equation can be applied to give accurate blood oxygen saturation readings. Other types of sensors, such as, indium gallium arsenide (InGaAs) detectors can be used for longer wavelengths, and lasers, for example, may be used for light emitters.

In some embodiments of this monitoring system, the sensor system (blood chamber 104, sensor unit 116, and emitter unit 118) may be arranged such that the blood chamber 104 is replaced by a section of tubing (for example, polyurethane) with repeatable sound characteristics that can be mass produced. The sensor unit 116 may then be replaced with a sound transducer, and the emitter unit 118 may be replaced with a sound emitter with ultra-sonic frequencies tuned to measure the viscosity or density of the blood. The acoustic measurement of the viscosity of blood can be equated to a level of hemoglobin content.

While optical and acoustic technologies are described for use in the sensor system, it can be appreciated that other types of sensors can be adapted to probe blood flowing from the body to measure various blood parameters for real-time monitoring without blood loss. In some embodiments, hybrid systems of different sensors are also possible.

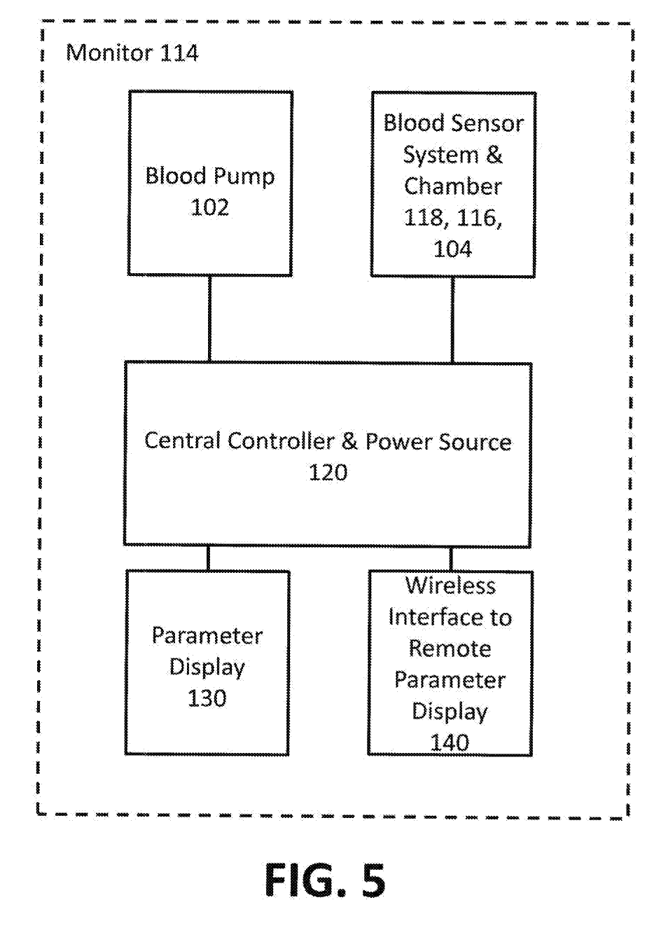

FIG. 5 illustrates an exemplary diagram of the blood monitor 114 controller system and power source 120. When used with surgical patients, the central controller and power source 120 is designed not to interfere with or not to be cumbersome to the patient or the clinical environment. The power source may be comprised of batteries such as one or more AA size cells. Due to the nature of an operating room in a healthcare facility or a hospital, the power source is designed to be sealed. The power source may be designed to be sealed for use in environments where gases are present. In some embodiments, the power source will be rechargeable. In some embodiments, when an external charging source is connected to the monitoring system 114, the external charging source will not only recharge the power source but also provide power to the monitor system 114. The power source may be constructed in multiple capacities and selected depending on length of the patient's procedure. In some embodiments, the power source is replaceable during the patient's procedure without data loss (a so called “hot swap”).

The central controller may include one or more processors or microcontrollers and non-transitory computer readable media with programmed instructions to perform tasks associated with managing the monitoring system 114. The central controller 120 manages the tasks of the monitor system 114. It will activate the blood pump 120 to bring blood from the patient to the blood sensor system and chamber. The blood sensor system identified as being, for example, in FIG. 1, the blood chamber 104, the sensor 116, and the emitter 118. The central controller 120 not only activates the blood pump 120, but may also determine and control the speed of the blood pump 102. The blood pump 102 may be powered by the power source.

The central controller and power source 120 also provides power and control signals to sensor elements 116 and 118 to manage which sensor elements in sensor 116 and which emitter elements in emitter 118 are turned ON and OFF. The central controller and power source 120 also determine the timing of measurement sampling, hence how frequent a measurement is taken. In an embodiment where the blood sensor system comprises one or more LED elements as emitter 118 and one or more photodetector elements as sensor 116, the transmitting LED(s) 118 and receiving photodetector(s) 116 are controlled by the central controller and power source 120. It is possible for some embodiments of this technology for the system to use continuous wave signal(s) as opposed to pulsed sampled signals.

The central controller and power source 120 can power and control a parameter display 130 in the form of a liquid crystal display (LCD) read-out or other form of graphical or text display. The data may be presented in either text or graphic format with calculations performed by the central controller 120 to drive the display.

In addition to or as an alternate method, the central controller and power source 120 can drive a wireless interface 140 communications link to a remotely located display. If attached to a surgical patient, the footprint of the entire monitoring system 114 may be miniaturized to the point of non-interference with clinical procedures and access to the patient. In such cases, an on-board display 130 may not be practical. Furthermore, a wireless link 140 using Bluetooth®, Wi-Fi, Zigbee® or other similar technology protocols can facilitate a large screen display located in a convenient and visible part of the clinical suite, ICU or operating room. The entire monitoring system 114 can remain small, out of the way, power independent and still produce valuable blood parameter and patient condition data on a large readable screen in the operating room. The monitoring system 114 may be moved to recovery where external power can be applied to the system and a display in that room may be updated to continue showing the history of the procedure.

The monitoring system 114 may be used in other situations not associated with surgery. It can be used with patients in the ICU suffering from any malady where observation of blood parameters in real time are of value in monitoring the patients' conditions.

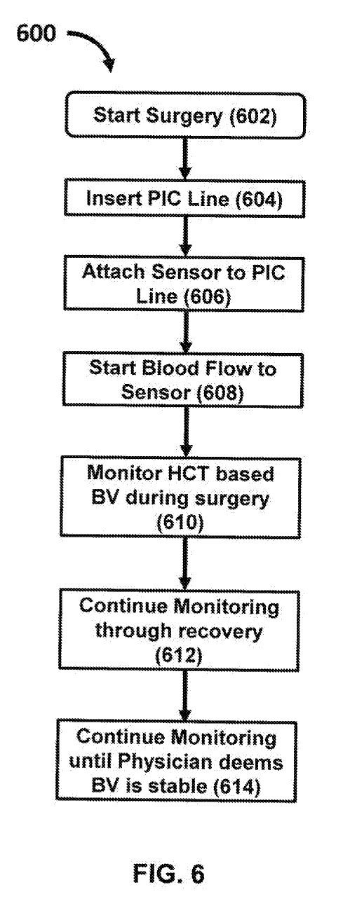

FIG. 6 is a flow diagram illustrating a process 600 of monitoring blood parameters using a monitoring system 114 according to some embodiments of the disclosure. Step 602 indicates the beginning of surgery. At step 604, a PIC line is inserted into the patient. The PIC line is either pre-installed or installed in the patient.

At step 606, the monitoring system 114 and the blood sensor system (104, 116, and 118) are connected to the PIC line connectors 16 and 26. In an embodiment, the monitoring system 114 operates on battery power, and the blood sensor system includes optical components. The blood pump 102 and the blood chamber 104 are attached to the arterial and venous ports of the PIC line, connectors 16 and 26, as appropriate. The optical emitter(s) 118 and optical sensor(s) 116 are seated onto the viewing area of the blood chamber 104.

At step 608, blood flow is started by the central controller and power source 120. The central controller engages the blood pump 102 to pump blood from the patient from the arterial port of the PIC line to the venous port of the PIC line. An extracorporeal tubing connecting both ports of the PIC line provides the monitoring system 114 access to the blood.

At step 610, one or more blood parameters are measured during surgery. For example, the blood sensor system (104, 116, and 118) obtains data on blood present in the blood chamber 104 by emitting light from the optical emitter(s) 118, having the emitted light pass through the blood in the blood chamber 104, and sensing the light received at the optical sensor(s) 116. Data obtained by the blood sensor system is processed by the central controller and power source 120 and may be transferred to a local display 130 (if installed in monitoring system 114) and/or sent wirelessly to a remote display to be viewed by individuals in the procedure room. In some embodiments, once data is being received by the central controller and power source 120 and verified as correct, the monitor system 114 is small enough to be placed out of the way, where it is unobtrusive during subsequent medical procedures. In an example, the blood parameter being measured is HCT, and from HCT values, change in blood volume is measured as surgery proceeds. A graphical screen may show the progress of blood volume changes over time. Monitoring of the change in blood volume during the surgery procedure can indicate to the surgical team how the procedure is advancing. For example, a sudden drop in blood volume could indicate unexpected blood loss.

At step 612, when the surgery is complete, the patient may be moved to recovery where the monitoring system 114 will remain in place and active. In recovery, effects of recovery medicines, such as, diuretic drugs, can be monitored to ensure that added fluids during surgery are being properly removed to return the patient's blood volume close to the patient's initial blood volume. While the patient is in recovery and in the post surgery phase, a small, low current charger may be attached to the monitoring system 114 to recharge the battery in the central controller and power source 120.

At step 614, the monitoring system 114 may be left in place until the physician is satisfied that the patient is stable, and there is no longer utility in monitoring the blood volume changes. HCT measurement and monitoring is used as an example to illustrate steps involved in process 600. It is understood that other parameters (including multiple parameters at the same time) can be monitored with the measurement system 114.

As examples, the blood monitoring system 114 can monitor loss of fluid from the intravascular compartment into the interstitial compartment and third spaces. That is, patient's progress and response to antibiotic therapy can be monitored to help optimize and minimize the complications of IV fluid therapy. In addition, the monitoring system 114 may be used for investigating new therapies introduced to treat septic states. Some other examples of measureable metrics include (but are not limited to): (1) Absolute HCT, and estimated hemoglobin which is useful to monitor for blood loss, anemia and patient response to transfusions; (2) Change in blood volume for evaluating third spacing in a sepsis situation, for detection of blood loss and/or evaluation of dialysis, CRRT and similar fluid management treatments for effectiveness; (3) Oxygen saturation is a key physiological parameter, which is a useful indicator for organ failure. The diagnostic capability of oxygen saturation depends on whether it is measured in arterial or venous blood. When measured in arterial blood, a low oxygen saturation is most frequently due to respiratory disorders. Low venous oxygen saturation is frequently seen with cardiac failure, in sepsis and major bleeds such as aortic aneurysm and rupture of the spleen; and (4) With the use of dye marker infusions into the patient's blood stream, parameters such as liver function can be determined.

Embodiments of the disclosure may be used to determine various real-time metrics indicative of a patient's body fluid condition. The real-time metrics may be determined using a diagnostic vascular window. The diagnostic vascular window may be created by installing low flow accesses to the patient's blood vessels, the low flow accesses including an arterial side access and a venous side access. A monitoring system according to some embodiments of the disclosure may be attached to the low flow accesses and blood may flow from the arterial side access to the venous side access. The monitoring system may then measure blood constituents from blood flowing from the arterial side access to the venous side access through the monitoring system. Since no treatment is being administered through the arterial side and venous side accesses of this window system and no treatment is being administered at the related monitoring system, the volume of fluid flowing out of the arterial side access during the course of a monitoring period is equal to a volume of fluid flowing back into the venous side access of the PIC line (it will be appreciated that the term “equal” is used herein to mean that the monitoring system is a closed loop circuit and that no fluid is added or removed due to treatment being performed via the extracorporeal blood being circulated out from arterial access). The monitoring period may be, for example, a period of time beginning when measurement begins and ending when measurement is stopped, or may be, for example, a period of time beginning when the accesses to the patient's blood vessels are connected and ending when the accesses to the patient's blood vessels are removed.

To the extent that treatment involving insertions into a patient's circulatory may be needed, such treatments may be administered through other accesses to the patient's circulatory system.

In one example, an extracorporeal tubing included in the monitoring system facilitates blood flow from the arterial side access to the venous side access of the PIC line, and the monitoring system attaches a blood sensor system to the extracorporeal tubing to measure blood parameters.

In another example, a blood chamber may be coupled to the low flow accesses using a blood chamber placed in the path of the tubing. The blood chamber provides a window where a blood sensor system of the monitoring system measures blood parameters.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for creating a diagnostic vascular window to monitor a patient's blood in real time, the method comprising:
installing low flow accesses to the patient's blood vessels, the low flow accesses comprising an arterial side access and a venous side access;
attaching a monitoring system to the low flow accesses;
starting blood flow to the monitoring system, the blood flowing from the arterial side to the venous side; and
measuring blood constituents from blood flowing through the monitoring system, wherein, during a monitoring period, a volume of fluid that flowed out of the arterial side access is equal to a volume of fluid that flowed into the venous side access.

2. The method according to claim 1, wherein attaching the monitoring system to the low flow accesses comprises:
attaching an extracorporeal tubing comprised in the monitoring system to the low flow accesses, wherein the extracorporeal tubing is configured to facilitate blood flow from the arterial side access to the venous side access; and
attaching a blood sensor system comprised in the monitoring system to the extracorporeal tubing.

3. The method according to claim 2, wherein the blood sensor system comprises one or more emitters and one or more sensors.

4. The method according to claim 3, wherein the one or more emitters are optical emitters and the one or more sensors are optical sensors, and the blood sensor system further comprises a blood chamber, the blood chamber configured to provide a location where blood within the blood chamber can be viewed using the optical emitters and the optical sensors.

5. The method according to claim 4, wherein the optical emitters are light emitting diodes (LEDs) or lasers.

6. The method according to claim 4, wherein the optical sensors are photodiodes.

7. The method according to claim 3, wherein the one or more emitters are acoustic emitters and the one or more sensors are acoustic sensors.

8. The method according to claim 1, wherein the monitoring system measures blood parameters comprising hematocrit, change in blood volume, and oxygen saturation.

9. The method according to claim 1, wherein the low flow accesses are peripherally inserted central catheter (PIC) lines or intravenous needles.

10. The method according to claim 1, wherein the low flow accesses support blood flowrates between 5 milliliters per minute and 50 milliliters per minute.

11. The method according to claim 1, wherein the low flow accesses support blood flowrates between 10 milliliters per minute and 20 milliliters per minute.

12. A system for monitoring a patient's blood in real time, the system comprising:
a blood pump configured to pump blood from an arterial side access to a venous side access, the arterial side access and the venous side access being low flow accesses;
tubing coupled to the blood pump, the tubing configured to carry extracorporeal blood from the arterial side access to the venous side access at a flowrate determined by the blood pump;
a blood sensor system coupled to the tubing, the blood sensor system configured to measure blood constituents of the extracorporeal blood flowing through the tubing;
wherein the system is configured such that, during a monitoring period, a fluid volume that flowed out from the arterial side access is equal to a fluid volume that flowed into the venous side access.

13. The system according to claim 12, wherein the blood sensor system comprises one or more emitters and one or more sensors.

14. The system according to claim 13, wherein the one or more emitters are optical emitters and the one or more sensors are optical sensors, and the blood sensor system further comprises a blood chamber, the blood chamber coupled to the tubing to provide a location where blood within the blood chamber can be viewed using the optical emitters and the optical sensors.

15. The system according to claim 14, wherein the optical emitters are light emitting diodes (LEDs) or lasers and the optical sensors are photodiodes.

16. The system according to claim 13, wherein the one or more emitters are acoustic emitters and the one or more sensors are acoustic sensors.

17. The system according to claim 12, wherein the low flow accesses are peripherally inserted central catheter (PIC) lines or intravenous needles.

18. The system according to claim 12, wherein the low flow accesses support blood flowrates between 5 milliliters per minute and 50 milliliters per minute.

19. The system according to claim 12, wherein the low flow accesses support blood flowrates between 10 milliliters per minute and 20 milliliters per minute.

20. The system according to claim 12, further comprising:
a controller configured to activate and determine speed of the blood pump; and
a power source configured to be replaceable, wherein the power source is selected based on a length of a medical procedure of the patient.
